(19) United States
(12) Patent Application Publication
(10) Pub. No.: US 2017/0224593 A1
(43) Pub. Date: Aug. 10, 2017

(54) COMPOSITION FOR MUD PACK AND MUD PACK COMPRISING THE SAME

(71) Applicant: Joo Won KIM, Hwaseong (KR)

(72) Inventor: Joo Won KIM, Hwaseong (KR)

(21) Appl. No.: 15/423,704

(22) Filed: Feb. 3, 2017

(30) Foreign Application Priority Data
Feb. 5, 2016 (KR) 10-2016-0015302

(51) Int. Cl. A61K 8/25; A61K 8/41; A61K 8/81; A45D 44/00; A61K 8/73; A61K 8/26; A61K 8/02; A61K 8/34; A61Q 19/00

(52) U.S. Cl. CPC A61K 8/25; A61K 8/345; A61K 8/41; A61K 8/8135; A61Q 19/00; A61K 8/73; A61K 8/26; A61K 8/0212; A45D 44/002; A61K 2800/48; A61K 2800/87; A61K 2800/5922

(57) ABSTRACT

The present invention provides a composition comprising a mud rich in various active ingredients such as natural minerals, wherein the composition has a sufficiently low viscosity so as to stably provide a sheet-type mud pack, and a mud pack comprising the same. The mud pack comprising the composition is excellent in sebum control ability and pore shrinking ability, and is very effective for improving skin smoothness.

### CROSS-REFERENCE TO RELATED APPLICATION

This application claims the benefit of Korean Patent Application No. 10-2016-0015302 filed on Feb. 5, 2016 in the Korean Intellectual Property Office, the disclosure of which is herein incorporated by reference in its entirety.

### TECHNICAL FIELD

The present invention relates to a composition having a low viscosity and capable of stably providing a sheet-type mud pack and to a mud pack containing the same.

### TECHNICAL FIELD

The present invention relates to a composition having a low viscosity and capable of stably providing a sheet-type mud pack and to a mud pack containing the same.

### BACKGROUND OF ART

A conventional mud mask pack was made into powder or cream shape, which was used by mixing with water or used by spreading and painting on a face using a cosmetic brush and then completely drying for about 30 to 60 minutes, followed by washing the face with water. However, there was an inconvenience that a powder formulation must be used by directly mixing because powder scattering is great, and a cream formulation must spread and paint using a brush. In addition, since the use time required to be dried after application is long, there was a problem that it is cumbersome to use the formulation alone.

### DETAILED DESCRIPTION OF THE INVENTION

### Technical Problem

It is an object of the present invention to provide a composition having a low viscosity and capable of stably providing a sheet-type mud pack.

It is another object of the present invention to provide a mud pack containing the above composition.

### Technical Solution

Hereinafter, a composition for a mud pack and a mud pack containing the same according to a specific embodiment of the present invention will be described.

According to one embodiment of the invention, there is provided a composition for a mud pack which contains a mud in an amount of 0.1 to 30% by weight based on the total weight of the entire composition, wherein the composition has a viscosity of 8,000 to 20,000 cps measured at 25° C.

Generally, the pack can be broadly classified into a wash-off type that washes off with water when the pack is dry, a peel-off type that peels off when a film is formed and dried, and a sheet-type in which a pack is attached to a face and, after a lapse of a certain time, the attached pack is removed. The type of pack is mainly determined according to cosmetic raw materials which are impregnated in the pack. And the mud is difficulty to stably apply to the mask sheet, and thus it was usually marketed as a wash-off type product.

Accordingly, the present inventors have conducted various tests to confirm the relationship between the composition and physical properties of the mud-containing composition, and also conducted various experiments to confirm the compatibility between the composition and the mask sheet, and the stability after the mud-containing composition was impregnated in the mask sheet, and the like. As a result, the inventors have found that a composition having a specific viscosity is useful for providing sheet-type mud packs, these mud packs are excellent in sebum control ability and pore-shrinking ability, and the use time is considerably shorter than conventional wash-off type mud packs, and the smoothness of skin after use is remarkably improved. The present invention has been completed on the basis of such finding.

The composition according to one embodiment of the invention may contain a mud in an amount of 0.1 to 30% by weight, 0.1 to 10% by weight, 0.1 to 5% by weight or 0.1 to 2% by weight, based on the total weight of the entire composition. Within these ranges, it is possible to provide a composition having a sufficiently low viscosity so as to stably provide a sheet-type mud pack while sufficiently exhibiting a skin beauty effect by the mud rich in various active ingredients such as natural minerals, that is, sebum control effect and pore shrinkage improvement effect.

The composition according to one embodiment of the invention may have a viscosity of 8,000 to 20,000 cps, 10,000 to 18,000 cps, or 12,000 to 16,000 cps measured at 25° C. Within these ranges, the composition is stably immersed in a mask sheet of various materials and can easily supply nutrition to the skin.

On the other hand, the composition according to one embodiment of the invention may further include various components known in the technical field to which the present invention pertains, in addition to the above-mentioned mud. As one example, the composition may further comprise a silicon-containing component, a surfactant, a moisturizer, a thickener, an emulsifier, a chelating agent, a skin conditioning agent, an antiseptic agent, a fragrance, a volcanic ash or a mixture thereof.

Specifically, the silicon-containing component may include one or more components selected from the group consisting of silicate and silica. More specifically, the silicate may include kaolin, bentonite, magnesium aluminum silicate or a mixture thereof. Such silicon-containing component can be contained in an amount of 5 to 30% by weight based on the total weight of the entire composition. If the content of the silicon-containing component is less than the above range, it can be separated because the properties are not stable. If the content of the silicon-containing component exceeds the above range, the viscosity becomes high and so it becomes difficult to allow the composition according to one embodiment to impregnate in the mask sheet.

Examples of the surfactant include glyceryl stearate, polysorbate, PEG-100 stearate, polyglyceryl-3-methyl glucose distearate, methyl glucose sesquistearate, glyceryl stearate SE, cetearyl alcohol, cetearyl glucoside or a mixture thereof. Such surfactant may be contained in an amount of 0.5 to 20% by weight based on the total weight of the entire composition.

As the moisturizer, one or more compounds selected from the group consisting of ethylene glycol, diethylene glycol, polyethylene glycol, propylene glycol, dipropylene glycol, polypropylene glycol, butylene glycol, glycerin, betaine and isomers thereof may be used. Such moisturizer may be contained in an amount of 1 to 10% by weight based on the total weight of the entire composition.

As the thickener, water-soluble polymers such as polyvinyl alcohol and sodium polyacrylate, gums such as xanthan gum, and mixtures thereof may be used. Such thickener may be contained in an amount of 0.1 to 10% by weight based on the total weight of the entire composition.

The chelating agent may include ethylene diamine tetraacetic acid, a salt thereof, or a mixture thereof. Such chelating agent may be contained in an amount of 0.001 to 2% by weight based on the total weight of the entire composition.

The skin conditioning agent may include hydrogenated polydecene, Trideceth-6, or a mixture thereof. Such skin conditioning agent may be contained in an amount of 0.001 to 2% by weight based on the total weight of the entire composition.

The antiseptic agent may include phenoxyethanol, ethylhexyl glycerin, 1,2-hexanediol, glyceryl caprylate, or a mixture thereof. Such antiseptic agent may be contained in an amount of 0.001 to 2% by weight based on the total weight of the entire composition.

The fragrance may be contained in an amount of 0.001 to 5% by weight based on the total weight of the entire composition. Further, the volcanic ash may be contained in an amount of 0.1 to 10% by weight based on the total weight of the entire composition.

The components of the composition according to one embodiment of the invention are not limited to those described above, and all components commonly used in the technical field to which the present invention pertains may be used.

However, it is possible to prepare a composition capable of stably providing a sheet-type mud pack by using 1 to 5% by weight of polyvinyl alcohol as a thickener based on the total weight of the entire composition.

The composition according to one embodiment of the invention includes the above-mentioned components, and it may further contain the remaining content of solvent. As the solvent, a water-soluble solvent such as water or alcohol can be used.

On the other hand, according to another embodiment of the invention, there is provided a mud pack comprising a mask sheet; and a composition according to one embodiment of the invention immersed in the mask sheet.

The composition according to one embodiment of the invention has excellent compatibility with mask sheets of various materials. Therefore, as the mask sheet, ail mask sheets of various materials known in the technical field to which the present invention pertains can be used. As an example, the mask sheet may include a cellulose-based polymer, a rayon, a polyester-based polymer, a nylon, a pure cotton, a naturally-derived pulp, a corn fabric, a nonwoven fabric obtained by mixing them, a nonwoven fabric obtained by laminating the fabrics made therefrom.

The sheet-type mud pack exhibits excellent sebum control ability and pore shrinking ability, and when peeled off after use, it is possible to remove corneocyte with low irritation similarly to a peel-off pack and make the skin smooth and healthy.

### Advantageous Effects

The present invention provides a composition comprising a mud rich in various active ingredients such as natural minerals, wherein the composition has a sufficiently low viscosity so as to stably provide a sheet-type mud pack. The mud pack comprising the above composition is excellent in sebum control ability and pore shrinking ability, and is very effective for improving skin smoothness.

### BRIEF DESCRIPTION OF THE DRAWING

FIG. 1 is a diagram schematically showing an apparatus and a process capable of providing a sheet-type mud pack according to an embodiment of the present invention.

### DETAILED DESCRIPTION OF THE EMBODIMENTS

Hereinafter, the operation and effect of the present invention will be described in more detail through specific examples. However, these examples are for illustrative purposes only and are not intended to limit the scope of the invention.

### EXAMPLE 1

### Preparation of Composition for Mud Pack and Mud Pack

(1) Preparation of Composition for Mud Pack

The raw materials 1 to 4 described in Table 1 below were sequentially introduced into a main kiln and dissolved by heating to 80° C. Subsequently, the raw materials 6 and 7 were dispersed in the raw material 5 and then introduced into the main kiln and dispersed. Then, the raw materials 8 to 11 were sequentially introduced into the main kiln and dispersed.

On the other hand, a part of purified water (raw material 1) was introduced into a separate first auxiliary kiln to which the raw material 12 was added and dispersed.

On the other hand, the raw materials 13 and 14 were sequentially introduced and dissolved at room temperature a separate second auxiliary kiln which is distinguished from the first auxiliary kiln.

Then, the mixture in the first auxiliary kiln was introduced into the main kiln and stirred. Subsequently, the mixture in the second auxiliary kiln was introduced into the main kiln and stirred. Next, the raw material 15 was introduced into the main kiln and thickened. The composition thus obtained was defoamed under vacuum and then stored. The composition of Example 1 below had a viscosity of 15,000 cps measured at 25° C.

TABLE 1

| | | | Comparative |
|---|---|---|---|
| | | Example 1 | Example 1 |
| NO | Name of raw materials | [unit: wt. %] | [unit: wt. %] |
| 1 | Purified water | 68.71 | 63.78 |
| 2 | Disodium EDTA | 0.02 | 0.02 |
| 3 | Polyvinyl alcohol | 3.00 | — |
| 4 | Betaine | 0.50 | 2.00 |
| 5 | Butylene glycol | 3.00 | 10.00 |
| 6 | Xanthan gum | 0.10 | 0.30 |
| 7 | Magnesium aluminum silicate | 0.50 | 0.50 |
| 8 | Mud | 1.00 | 3.00 |
| 9 | Bentonite | 3.00 | 1.00 |
| 10 | Kaoline | 10.00 | 15.00 |
| 11 | Volcanic ash | 4.50 | 3.50 |
| 12 | Polysorbate | 5.00 | — |
| 13 | Phenoxyethanol | 0.35 | 0.30 |
| 14 | Fragrance | 0.07 | 0.10 |
| 15 | Sodium polyacrylate/ | 0.25 | 0.50 |
| | hydrogenated polydecene | | |
| | Trideceth-6 | | |

(2) Preparation of Sheet-Type Mud Pack

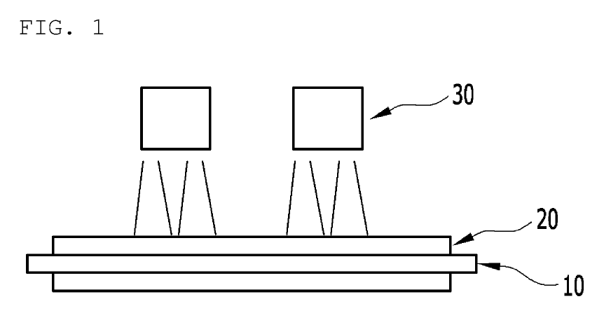

As shown in FIG. 1, the composition prepared above was impregnated in a nonwoven fabric. Then, the non-woven fabric impregnated with the above composition was doped through a doping machine and then pierced to prepare a mud pack.

### COMPARATIVE EXAMPLE 1

### Preparation of Liquid-Type Mud Pack

The raw materials 1, 2 and 4 described in Table 1 were sequentially introduced into the main kiln and dissolved by heating up to 80° C. Subsequently, the raw materials 6 and 7 were dispersed in the raw material 5, and introduced into the main kiln and dispersed. Then, the raw materials 8 to 11 were sequentially introduced into the main kiln and dispersed.

On the other hand, the raw materials 13 and 14 were sequentially introduced into a separate auxiliary kiln and dissolved.

Then, the mixture in the auxiliary kiln was introduced into the main kiln and stirred. Subsequently, the raw material 15 was introduced into the main kiln and thickened. The composition thus obtained was defoamed under vacuum and then stored. The composition of Comparative Example 1 below had a viscosity of 40,000 cps measured at 25° C.

Method of Using the Mud Pack Produced in Example 1 and Comparative Example 1

The sheet-type mud pack of Example 1 was attached to the user's face. After about 15 to 30 minutes, the mask sheet was removed, and washed with water.

On the other hand, the liquid-type mud puck of Comparative Example 1 was evenly applied to the face of the user, followed by washing with water after about 30 to 60 minutes.

### TEST EXAMPLE 1

### Evaluation of Sebum Control

In order to evaluate the sebum control ability of the mud pack according to one embodiment of the present invention, 25 healthy female subjects were allowed to use the mud pack prepared according to Example 1 and Comparative Example 1, respectively.

Then, the degree of satisfaction of each mud pack was investigated for the 25 subjects. The number of the subjects who answered that the mud pack of Example 1 was more satisfactory was described in Example 1, and the number of the subjects who answered that the mud pack of Comparative Example 1 was more satisfactory was described in Comparative Example 1.

TABLE 2

| | Example 1 | Comparative Example 1 |
|---|---|---|
| Satisfaction | 22 | 3 |

The sheet-type mud pack prepared according to Example 1 exhibited a dark gray color before drying and a gray color after drying. In addition, a portion with high sebum secretion exhibited a dark gray color even after drying. When the sheet-type mud pack of Example 1 was used for each subject, the time for changing the color of the mud pack was measured. As a result of the measurement, there were individual differences, but with a time lapse of about 15 to 25 minutes after the attachment of the mud pack, the mud pack color of the T zone where the sebum is much distributed exhibited a dark gray, and the mud pack color of the U zone where the sebum is relatively less distributed exhibited a light gray. Thus, when the sheet-type mud pack prepared according to Example 1 was used, the sebum secretion site and secretion degree could be confirmed.

In contrast, in the case of using the liquid-type mud pack prepared according to Comparative Example 1, the mud pack was dried with a time lapse of about 50 to 60 minutes after the application to the faces of the subjects, and a change in color was observed. Further, when the liquid-type mud pack prepared according to Comparative Example 1 was used, it was impossible to confirm the area where the sebum was swollen and the degree of sebum secretion.

### TEST EXAMPLE 2

### Evaluation of Pore Shrinkage Effect

In order to confirm the pore shrinkage effect of the mud pack according to one embodiment of the present invention, 22 healthy female subjects were allowed to use the mud packs prepared according to Example 1 and Comparative Example 1, respectively.

Then, the number of skin pores in the subjects before and after use of the mud packs was measured by the following method, and the results are shown in Table 3 below.

First, the subjects were rested for 30 minutes in a waiting room under constant temperature and humidity conditions with a room temperature of 20 to 25° C. and a humidity of 40 to 60%, and water ingestion was restricted during this time.

Then, using a skin pore measuring device (Visioface Quick), the facial image was photographed with respect to the cheek part of the subject, and the number of pores was measured. After repeatedly photographing the same site several times, the number of pores was defined as the average value.

TABLE 3

| | Example 1 | Comparative Example 1 |
|---|---|---|
| Before use of mud | 33.00 | 34.00 |
| pack | | |
| After of use of mud | 23.56 | 29.86 |
| pack | | |
| Variation rate[%] | −28.6% | −12.17% |

Variation rate of the number of pores (%)=[{(Number of pores after use of mud pack)−(number of pores before use of mud pack)}/(number of pores before use of mud pack)]*100

Referring to Table 3 above, it is confirmed that, when the sheet-type mudpack of Example 1 was used, the pore shrinkage effect was superior to that of the liquid-type mud pack of Comparative Example 1.

### TEST EXAMPLE 3

### Evaluation of Skin Smoothness

In order to evaluate the degree of skin smoothness after use of the mud pack according to one embodiment of the present invention, 22 healthy female subjects were allowed to use the mud pack prepared according to Example 1 and Comparative Example 1, respectively.

Then, the degree of satisfaction of each mud pack was investigated for the 22 subjects. The number of the subjects who answered that the skin was smoother after use of the mud pack of Example 1 was described in Example 1, and the number of the subjects who answered that the skin was smoother after use of the mud pack of Comparative Example 1 was described in Comparative Example 1.

TABLE 4

| | Example 1 | Comparative Example 1 |
|---|---|---|
| Degree of | 20 | 2 |
| satisfaction of | | |
| skin smoothness | | |

Referring to Table 4 above, it is confirmed that the mud pack of Example 1 had a higher degree of satisfaction of the skin smoothness after use as compared with the mud pack of Comparative Example 1. These results are expected to be shown because the sheet-type mud pack of Example 1 had a corneocyte-removing effect when peeling off from a face after drying.

### TEST EXAMPLE 4

### Evaluation of Drying Speed

In order to evaluate the drying speed of the mud pack according to one embodiment of the present invention, 20 healthy female subjects were allowed to use the mud pack prepared according to Example 1 and Comparative Example 1, respectively. And the average value of the drying time for each mud pack was described in Table 5 below.

TABLE 5

| | Example 1 | Comparative Example 1 |
|---|---|---|
| Drying time | 25 minutes | 50 minutes |

In addition, the preference for adhesiveness and smoothness after use of the mud packs of Example 1 and Comparative Example 1 was investigated for 20 subjects, and the scores of each item were averaged and shown in Table 6 below.

The subjects responded as: “4 points” if the adhesiveness and smoothness were very satisfactory, “3 points” if they were slightly satisfactory, “2 points” if they were good, and “0 points if they were bad.

TABLE 6

| | Example 1 | Comparative Example 1 |
|---|---|---|
| Adhesiveness | 3.85 | 3.10 |
| during use | | |
| Smoothness after | 4.15 | 3.25 |
| use | | |

### TEST EXAMPLE 5

### Evaluation of Nonwoven Fabric Compatibility

In order to evaluate the compatibility of the mud pack according to one embodiment of the present invention with the nonwoven fabric, the composition prepared in Example 1 was immersed in the nonwoven fabrics of various materials listed in Table 7 below to evaluate the compatibility.

TABLE 7

| | | Degree of | |
|---|---|---|---|
| | | maintaining | |
| | Degree of | nonwoven fabric | Drying |
| Classification | immersion | when immersed | speed |
| Cellulose type | ◯ | ◯ | Δ |
| Long fiber(hole type) | ⊚ | ⊚ | ⊚ |
| Rayon | Δ | Δ | Δ |
| Rayon + polyester | ◯ | ◯ | ◯ |
| Rayon + polyester(hole | ⊚ | ⊚ | ⊚ |
| type) | | | |
| Naturally-derived pulp | Δ | Δ | Δ |
| Corn fabric | ◯ | ◯ | ◯ |

On the other hand, the compositions prepared in Comparative Example 1 were also immersed in the nonwoven fabrics of various materials listed in Table 7 to evaluate the compatibility.

TABLE 8

| | | Degree of | |
|---|---|---|---|
| | | maintaining | |
| | Degree of | nonwoven fabric | Drying |
| Classification | immersion | when immersed | speed |
| Cellulose type | Δ | Δ | X |
| Long fiber(hole type) | Δ | Δ | Δ |
| Rayon | X | X | X |
| Rayon + polyester | Δ | Δ | Δ |
| Rayon + polyester(hole | X | X | X |
| type) | | | |
| Naturally-derived pulp | X | X | X |
| Corn fabric | X | X | X |

The symbols in Tables 7 and 8 have the following meanings.

⊚: Very Excellent, ◯: Excellent, Δ: Normal, ×: Poor

Specifically, in the item “degree of immersion”, if the composition is evenly immersed in the whole nonwoven fabric, it is evaluated as “very excellent”; if the composition is fully adhered on the nonwoven fabric but the deviation is partially observed, it is evaluated as “excellent”; if the amount remaining in the pouch is greater than the amount adhered on the nonwoven fabric or if it is not partially immersed, it is evaluated as “normal”; and if it does not fall under the above cases, it is evaluated as “poor”.

In the item “degree of maintaining nonwoven fabric when immersed”, the degree of maintaining the shape of the fabric when attached to the face after immersion was evaluated. If the mud pack is completely adhered to the face and does not detach until use, it is evaluated as “very excellent”; if the mud pack is closely adhered to the face but it is detached without being maintained during the use time, it is evaluated as “excellent”; if there is a part that is floating without being adhered to the face, it is evaluated as “normal”; and if it does not fall under the above cases, it is evaluated as “poor”.

In the item “drying speed”, if the drying time is 30 minutes or less, it is evaluated as “very excellent”; if the drying time is 45 minutes or less, it is evaluated as “excellent”; if the drying time is 60 minutes or less, it is evaluated as “normal”; and if it does not fall under the above cases, it is evaluated as “poor”.

Referring to Tables 7 and 8 above, it is confirmed that the composition prepared according to Example 1 can be applied to a wide variety of nonwoven fabrics compared to the composition of Comparative Example 1.

### EXPLANATION OF REFERENCE NUMERALS

10: Support (nonwoven fabric)

20: composition for mud pack

30: Doping machine

1. A composition for a mud pack which contains a mud in an amount of 0.1 to 30% by weight based on the total weight of the entire composition, wherein the composition has a viscosity of 8,000 to 20,000 cps measured at 25° C.

2. The composition for a mud pack according to claim 1, further comprising one or more silicon-containing components selected from the group consisting of silicate and silica.

3. The composition for a mud pack according to claim 2, wherein the silicon-containing component is contained in an amount of 5 to 30% by weight based on the total weight of the entire composition.

4. The composition for a mud pack according to claim 1, further comprising a surfactant.

5. The composition for a mud pack according to claim 4, wherein the surfactant is contained in an amount of 0.5 to 20% by weight based on the total weight of the entire composition.

6. The composition for a mud pack according to claim 1, further comprising a moisturizer, a thickener, an emulsifier, a chelating agent, a skin conditioning agent, an antiseptic agent, a fragrance, a volcanic ash or a mixture thereof.

7. The composition for a mud pack according to claim 6, wherein the moisturizer includes one or more compounds selected from the group consisting of ethylene glycol, diethylene glycol, polyethylene glycol, propylene glycol, dipropylene glycol, polypropylene glycol, butylene glycol, glycerin, betaine and isomers thereof.

8. The composition for a mud pack according to claim 6, wherein the moisturizer is contained in an amount of 1 to 10% by weight based on the total weight of the entire composition.

9. The composition for a mud pack according to claim 6, wherein the thickener includes water-soluble polymers, gums or mixtures thereof.

10. The composition for a mud pack according to claim 6, wherein the thickener is contained in an amount of 0.1 to 10% by weight based on the total weight of the entire composition.

11. The composition for a mud pack according to claim 6, wherein the chelating agent includes ethylene diamine tetraacetic acid, a salt thereof, or a mixture thereof.

12. The composition for a mud pack according to claim 6, wherein the chelating agent is contained in an amount of 0.001 to 2% by weight based on the total weight of the entire composition.

13. The composition for a mud pack according to claim 6, wherein the composition includes 1 to 5% by weight of polyvinyl alcohol as a thickener based on the total weight of the entire composition.

14. A mud pack comprising a mask sheet; and
the composition according to claim 1 immersed in the mask sheet.

15. The mud pack according to claim 14, wherein the mask sheet includes a cellulose-based polymer, a rayon, a polyester-based polymer, a nylon, a pure cotton, a naturally-derived pulp, a corn fabric, a nonwoven fabric obtained by mixing them, a nonwoven fabric obtained by laminating the fabrics made therefrom.
